(19) United States
(12) Patent Application Publication
(10) Pub. No.: US 2009/0197311 A1
(43) Pub. Date: Aug. 6, 2009

(54) METHOD FOR THE PRODUCTION OF SIMVASTATIN

(76) Inventors: Marco Alexander Van Den Berg, Poeldijk (NL); Marcus Hans, Den Haag (NL); Hugo Streekstra, Amsterdam (NL)

Correspondence Address: NIXON & VANDERHYE, PC, 901 NORTH GLEBE ROAD, 11TH FLOOR, ARLINGTON, VA, 22203, US

(21) Appl. No.: 12/304,283

(22) PCT Filed: Jun. 18, 2007
(86) PCT No.: PCT/EP2007/056014

§ 371 Date: Dec. 11, 2008

(30) Foreign Application Priority Data
Jun. 20, 2006 (EP) 06115721.0

(51) Int. Cl. C12P 17/06; C12N 1/21; C12N 1/15; C07H 21/04; C07K 14/00

(52) U.S. Cl. 435/125; 435/252.31; 435/252.33; 435/254.3; 435/254.5; 536/23.7; 530/350

(57) ABSTRACT

The present invention provides a fermentative process for the synthesis of simvastatin by providing a host capable of incorporating the 2,2-dimethylbutyrate side chain into simvastatin, i.e. by customizing a polyketide synthase gene optimized for synthesis and/or incorporation of 2,2-dimethylbutyrate; optionally feeding said host with the appropriate substrate for 2,2-dimethylbutyrate synthesis; fermenting said host to obtain simvastatin or analogues or derivatives thereof, i.e. by producing simvastatin on an industrial scale by a fed-batch process.

### FIELD OF THE INVENTION

The present invention relates to the fermentative production of the HMG-CoA reductase inhibitor simvastatin in a host cell.

### BACKGROUND OF THE INVENTION

Cholesterol and other lipids are transported in body fluids by low-density lipoproteins (LDL) and high-density lipoproteins (HDL). Substances that effectuate mechanisms for lowering LDL-cholesterol may serve as effective antihypercholesterolemic agents because LDL levels are positively correlated with the risk of coronary artery disease. Cholesterol lowering agents of the statin class are medically very important drugs as they lower the cholesterol concentration in the blood by inhibiting HMG-CoA reductase. The latter enzyme catalyses the rate limiting step in cholesterol biosynthesis, i.e. the conversion of (3S)-hydroxy-3-methylglutarylcoenzyme A (HMG-CoA) to mevalonate. Today, simvastatin is amongst the most prescribed drugs in cholesterol lowering applications. The synthesis of simvastatin is not straightforward as it involves a semi-synthetic approach starting from the natural product lovastatin (synthesized by Aspergillus terreus).

The molecular difference between lovastatin and simvastatin resides in the side chain on the C-8 position. Here, lovastatin carries a 2-methylbutyrate moiety, while simvastatin has a 2,2-dimethylbutyrate moiety on this position. Numerous chemical syntheses of simvastatin have been reported worldwide since its discovery in 1984. One route, as described in U.S. Pat. No. 4,444,784, involves de-esterification of the 2-methylbutyrate side chain of lovastatin, followed by several distinct chemical steps that involve lactonization, hydroxy group protection/deprotection and re-esterification with the appropriate 2,2-dimethylbutyrate side chain. This process results in a low overall yield. Another method, described in U.S. Pat. No. 4,582,915, involves direct methylation of the lovastatin side chain using a metal alkyl amide and methyl halide. This method suffers from problems concerning the purity of the final product as it leads to several by-products that have to be separated from the target compound. Furthermore, the chemicals used are toxic and/or carcinogenic making implementation on an industrial scale difficult and hazardous. Likewise, yet another method described in U.S. Pat. No. 4,820,850, while addressing the problems of low overall yield and purity, involves as many as six chemical steps that also utilize reagents that are unsafe to handle on an industrial scale. The more recent method described in U.S. Pat. No. 5,763,646 involves conversion of lovastatin to simvastatin using fewer chemical steps. However, this method employs expensive chemical reagents and also results in low overall yield.

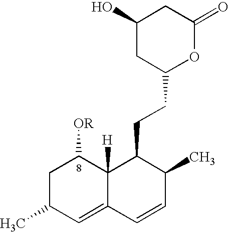

| Name | R |
|---|---|
| Lovastatin | 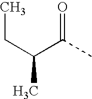 |
| Monacolin J | H |
| Simvastatin | 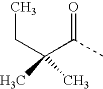 |

As an alternative for the existing chemical routes WO 03/010324 describes the metabolic engineering of one of both polyketide synthases (LovF) involved in the biosynthesis of lovastatin. However, the problem underlying the direct fermentative production of simvastatin in a host is the incorporation of the unnatural side chain, 2,2-dimethylbutyrate. By exchanging modules of the LovF polyketide synthase for modules from other sources this problem is not solved because, in order to synthesize the 2,2-dimethylbutyrate side chain, the modified LovF enzyme requires a substrate (methylmalonyl-CoA) that is not present in lovastatin producing organisms such as Aspergillus terreus. In WO 00/037629 it is suggested to modify the lovB gene for the production of other HMG-CoA reductase inhibitors. As the lovB gene is involved in the biosynthesis of the monacolin J core indeed other HMG-CoA reductase inhibitors are accessible through modification of this gene, however not simvastatin as the formation of the side chain at position C-8 does not reside in the lovB gene.

### SUMMARY OF THE INVENTION

It is an object of the present invention to provide a fermentative process for simvastatin synthesis thereby avoiding the complex synthetic steps required to convert lovastatin and overcoming the problem associated with the disclosure of WO 03/010324.

The present invention solves the problem outlined above by providing a host with the necessary building blocks for the in vivo synthesis of the 2,2-dimethylbutyrate-side chain/on simvastatin. As is shown any species with a gene cluster consisting of one or more lovastatin biosynthetic genes can produce simvastatin as either methylmalonate, methylmalonyl-CoA or 2,2-dimethylbutyrate is provided to the host cell. Apart from 2,2-dimethylbutyrate itself also derivatives thereof, such as thio-esters, can be used. This can be done by means of external feeding or, alternatively, by means of engineering the host cell with a methylmalonyl-CoA synthesis pathway. The other building block, monacolin J, is either produced by or fed to this cell containing one or more lovastatin biosynthetic genes. Thus, the above and any other objects of the invention are achieved by the method of the present invention, wherein said method comprises:
providing a host capable of incorporating the 2,2-dimethylbutyrate side chain into simvastatin, i.e. by customizing a polyketide synthase gene optimized for synthesis and/or incorporation of 2,2-dimethylbutyrate
fermenting said host to obtain simvastatin or analogues or derivatives thereof, i.e. by producing simvastatin on an industrial scale by a fed-batch process

Embodiments of the invention relate to methods for feeding the host with all forms of methylmalonate or 2,2-dimethylbutyrate or derivatives of these compounds such as thio-esters to obtain the 2,2-dimethylbutyrate side chain intracellular and/or engineering the host with metabolic pathways capable of in vivo synthesis of methylmalonate-CoA. The methylmalonate-CoA biosynthetic pathway may consist of two enzymes, namely propionyl-CoA synthetase and propionyl-CoA carboxylase, wherein the two enzyme pathway is obtained from Aspergillus nidulans (propionyl-CoA synthetase) and Streptomyces coelicolor (propionyl-CoA carboxylase) and wherein the two enzymes are selected from any available propionyl-CoA synthetase and propionyl-CoA carboxylase homologous genes in nature, plus propionate feeding. Alternatively, the methylmalonate biosynthetic pathway consists of one enzyme, namely malonyl-CoA synthetase, wherein the one enzyme pathway is obtained from Rhizobium species, and/or wherein the one enzyme is selected from any available malonyl-CoA synthetase homologous gene in nature. An example of efficient methylmalonyl-CoA engineering is published by Reeves et al. (2007; Metabol. Engineer. 9:293-303). The host is any host equipped with one or more genes encoding the lovastatin biosynthetic machinery and preferably is a eukaryote selected from the group of fungi, preferably selected from the species Aspergillus, Penicillium or Saccharomyces.

### DETAILED DESCRIPTION OF THE INVENTION

The term lovastatin biosynthetic gene encompasses any of the wild type genes from Aspergillus terreus, including also modified, inactive and truncated variants plus homologous enzymes and non-homologous enzymes with the same function (i.e. the lovastatin enzymes system).

The term 2,2-dimethylbutyrate encompasses all bio-available molecules containing the CH₃CH₂C(CH₃)₂C—R moiety, in which R can be OH, O⁻X⁺ wherein X⁺ represents a cation such as a metal cation, ammonia or other nitrogen derived cations. Particularly suitable compounds are those wherein R represents an activated group. Any activated group known to the skilled person is suitable. Particularly suitable activated groups are for instance S-coenzyme A (SCoA) or derivatives thereof, S-N-acetylcysteamine (SNAC) or derivatives thereof, S-methylthioglycolate (SMTG), thioalkyl groups and the like.

The first aspect of this invention is to equip the host with a steady supply of the 2,2-dimethylbutyrate-side chain. Using LC-MS analysis it was demonstrated that this compound is not synthesized or present in natural lovastatin producers (e.g. Aspergillus terreus). Secondly, in wild type Aspergillus terreus, grown under lovastatin producing conditions, neither 2,2-dimethylbutyrate nor simvastatin can be detected, intra-or extracellularly. Thirdly, enzymatic measurements have shown that methylmalonyl-CoA, the presumed precursor for 2,2-dimethylbutyrate synthesis, is also not present in wild type lovastatin producing Aspergillus terreus. Taken this together, it is shown that Aspergillus terreus lacks methylmalonyl-CoA to synthesize 2,2-dimethylbutyrate.

One embodiment of the invention describes the feeding of methylmalonyl-CoA to a cell-free extract of an organism (e.g. Aspergillus terreus) that harbors the complete set or single genes of the lovastatin biosynthetic gene cluster, or any other organism that is capable of producing lovastatin by means of genetic engineering. Suitable organisms are prokaryotes chosen from the group consisting of Bacillus amolyquefaciens, Bacillus subtilis and Escherichia coli or eukaryotes chosen from the group consisting of Aspergillus nidulans, Aspergillus terreus, Aspergillus niger, Penicillium citrinum, Penicillium brevicompactum, Penicillium chrysogenum, Monascus ruber, Monascus purpurea, Saccharomyces cerevisiae and Kluyveromyces lactis. The organism is grown under lovastatin producing conditions as described in WO 98/37179. Alternatively, the level of lovatstatin and/or intermediates can be increased by external feeding. In the presence of other essential building blocks and co-factors (such as monacolin J, acetyl-CoA, NADPH, ATP and S-adenosylmethionine) the lovastatin biosynthetic enzymes surprisingly can use methylmalonyl-CoA to synthesize simvastatin. Hence it was demonstrated that the wild type Aspergillus terreus is not capable of synthesizing methylmalonate, methylmalonyl-CoA and/or 2,2-dimethylbutyrate.

Another embodiment of the invention describes the feeding of the simvastatin precursor 2,2-dimethylbutyrate to a culture of an organism (e.g. Aspergillus terreus) that harbors the complete set or single genes of the lovastatin biosynthetic gene cluster, or any other organism that is capable of producing lovastatin by means of genetic engineering (e.g. Penicillium chrysogenum, Saccharomyces cerevisiae, Bacillus subtilis, Escherichia coli) growing under lovastatin producing conditions (see WO 98/37179). The organism can harbor part of (as demonstrated by Xie et al. in Chemistry & Biology 13, 1161 (2006) and in Appl. Environ. Microbiol. 73, 2054 (2007)) or the complete set of lovastatin biosynthetic genes or modified/inactivated lovastatin biosynthetic genes, also, biosynthetic genes that are homologous to the lovastatin biosynthetic genes (30-40% identical on amino acid level is considered to be homologous in this context). A polypeptide having an amino acid sequence that is “substantially homologous” to the lovastatin biosynthetic genes is defined as a polypeptide having an amino acid sequence possessing a degree of identity to the specified amino acid sequence of at least 30%, preferably at least 40%, more preferably at least 50%, still more preferably at least 60%, still preferably at least 70%, still more preferably at least 80%, still more preferably at least 90%, still more preferably at least 98% and most preferably at least 99%, the substantially homologous peptide displaying activity towards the synthesis of lovastatin and/or simvastatin. A substantially homologous polypeptide may encompass polymorphisms that may exist in cells from different populations or within a population due to natural allelic or intra-strain variation. A substantially homologous polypeptide may further be derived from a species other than the fungus where the specified amino acid and/or DNA sequence originates from, or may be encoded by an artificially designed and synthesized DNA sequence. DNA sequences related to the specified DNA sequences and obtained by degeneration of the genetic code are also part of the Invention. Homologues may also encompass biologically active fragments of the full-length sequence. For the purpose of the present invention, the degree of identity between two amino acid sequences refers to the percentage of amino acids that are identical between the two sequences. The degree of identity is determined using the BLAST algorithm, which is described in Altschul, et al., J. Mol. Biol. 215: 403-410 (1990). Software for performing BLAST analyses is publicly available through the National Center for Biotechnology Information (http://www.ncbi.nim.nih.gov/). The BLAST algorithm parameters W, T, and X determine the sensitivity and speed of the alignment. The BLAST program uses as defaults a word length (W) of 11, the BLOSUM62 scoring matrix (see Henikoff & Henikoff, Proc. Natl. Acad. Sci. USA 89: 10915 (1989)) alignments (B) of 50, expectation (E) of 10, M=5, N=−4, and a comparison of both strands.

Substantially homologous polypeptides may contain only conservative substitutions of one or more amino acids of the specified amino acid sequences or substitutions, insertions or deletions of non-essential amino acids. Accordingly, a non-essential amino acid is a residue that can be altered in one of these sequences without substantially altering the biological function. For example, guidance concerning how to make phenotypically silent amino acid substitutions is provided in Bowie, J. U. et al., Science 247:1306-1310 (1990) wherein the authors indicate that there are two main approaches for studying the tolerance of an amino acid sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. As the authors state, these studies have revealed that proteins are surprisingly tolerant of amino acid substitutions. The authors further indicate which changes are likely to be permissive at a certain position of the protein. For example, most buried amino acid residues require non-polar side chains, whereas few features of surface side chains are generally conserved. Other such phenotypically silent substitutions are described in Bowie et al, and the references cited therein.

The term “conservative substitution” is intended to mean that a substitution in which the amino acid residue is replaced with an amino acid residue having a similar side chain. These families are known in the art and include amino acids with basic side chains (e.g. lysine, arginine and histidine), acidic side chains (e.g. aspartic acid, glutamic acid), uncharged polar side chains (e.g., glycine, asparagines, glutamine, serine, threonine, tyrosine, cysteine), non-polar side chains (e.g., alanine, valine, leucine, isoleucine, proline, phenylalanine, methionine, tryptophan), β-branched side chains (e.g., threonine, valine, isoleucine) and aromatic side chains (e.g., tyrosine, phenylalanine tryptophan, histidine).

Additionally, biosynthetic gene clusters that are not homologous, but follow the same biosynthetic building principle for statin synthesis can be used. When 2,2-dimethylbutyrate was fed to an organism with one or more lovastatin biosynthetic genes, for example an intact diketide synthase gene (such as the lovF gene), a mixture of lovastatin and simvastatin was obtained, roughly in a ratio of 50:1. The lovastatin biosynthetic gene lovF from Aspergillus terreus was described as a gene encoding an enzyme that produces the 2-methylbutyrate moiety (Hutchinson, C. R. et al., Antonie van Leeuwenhoek 2000, 3-4, pages 287-295). So, surprisingly, the lovastatin enzyme system exhibits, besides the natural preference for 2-methylbutyrate, a relatively high substrate tolerance towards 2,2-dimethylbutyrate. A further improvement can be achieved by addition of a lovF gene optimized by methods known in the art (e.g. directed evolution, gene shuffling or side directed mutagenesis) with a preference for synthesizing 2,2-dimethylbutyrate and/or inactivating the wild type lovF gene of Aspergillus terreus by inserting a point mutation (artificial stop codon). Feeding of 2,2-dimethylbutyrate to a microorganism harboring the lovastatin biosynthetic genes resulted in improved simvastatin production.

A further embodiment describes the engineering of potential hosts, such as Aspergillus terreus, with pathways for in vivo methylmalonyl-CoA production. This can be done in three ways. The first route starts from propionate. Propionate is converted to propionyl-CoA and subsequently carboxylated to methylmalonyl-CoA. The enzymes for this pathway can be obtained from Streptomyces coelicolor (Diacovich, L. et al., J. Biol. Chem. 2002, 41, pages 31228-31236) or any other species harboring homologous genes. Aspergillus terreus hosts equipped with this pathway need a propionate feed during fermentation for optimal methylmalonyl-CoA synthesis.

In yet another embodiment, use is made of malonyl-CoA synthetase from Rhizobium sp. (Kim, Y. S. et al., Biochem. J. 1991, 273, pages 511-516), which normally catalyzes the formation of malonyl-CoA from malonate. As described In Pohl, N. L. et al., J. Amer. Chem. Soc. 2001, 123, pages 5822-5823, malonyl-CoA synthetase has an unusually high substrate tolerance, and easily converts methylmalonate into the corresponding CoA ester with comparable rates to the wild type reaction. Therefore, integration of the malonyl-CoA synthetase gene and external feeding of methylmalonate leads to an alternative way for in vivo methylmalonyl-CoA production.

In yet another embodiment, use is made of the methylmalonyl-CoA mutase-epimerase pathway (Dayem, L. C. et al., Biochemistry 2002, 41, pages 5193-5201). This involves the sequential actions of two enzymes, methylmalonyl-CoA mutase and methylmalonyl-CoA epimerase, which convert succinyl-CoA to (2R)- and then to (2S)-methylmalonyl-CoA. When cells harboring Propionibacterium shermanii methylmalonyl-CoA mutase and Streptomyces coelicolor methylmalonyl-CoA epimerase are fed with the B12 precursor hydroxycobalamin and propionate methylmalonyl-CoA is produced.

The second aspect of this invention is to equip the host with a polyketide synthetase and/or other enzymes of the lovastatin enzyme system that is optimized for synthesizing and/or attaching this 2,2-dimethylbutyrate to the monacolin J core as compared to the low activity of the natural enzymes.

As described above besides the commonly accepted side chain 2-methylbutyrate, the modified LovF protein can also synthesize the simvastatin side chain 2,2-dimethylbutyrate, although in lower yields. Modified LovF belongs to the enzyme class of fungal polyketide synthases. A remarkable feature of polyketide synthethases is the domain architecture. In bacterial type I polyketide synthases (Khosla, C. et al., Chem. Rev. 1997, 97, pages 2577-2590) domains are organized within modules, and each module only catalyzes one condensation reaction. In fungal systems, the situation is more difficult. Fungal polyketide synthases are large proteins with multiple domains. Moreover, in many described cases, the domains seem to be used multiple times, e.g. an enzyme with only one keto-synthase domain produces a nonaketide (see for example Hutchinson, C. R. et al., Antonie van Leeuwenhoek 2000, 3-4, pages 287-295). The mechanism behind this is not fully understood. In contrast, the LovF enzyme of Aspergillus terreus seems to be the simplest case of a fungal polyketide synthase. It consists of a single polypeptide comprised of six domains. Remarkably, each domain is used only once, giving rise to 2-methylbutyrate. To optimize the yield of a simvastatin fermentation using a host capable of synthesizing methylmalonyl-CoA in vivo, the LovF protein needs to be optimized for using this substrate and convert it into 2,2-dimethylbutyrate. With this respect, the simple architecture is helpful as it functions comparable to a type I PKS. This is a prerequisite for engineering the enzyme towards the 2,2-dimethylbutyrate polyketide synthase.

In one embodiment, the LovF enzyme was engineered by replacing several domains, which led to an increased production of 2,2-dimethylbutyrate from methylmalonate-CoA, and, when integrated into the lovastatin biosynthetic gene cluster (replacing the wild type lovF gene) of a methylmalonyl-CoA producing host, an increased production of simvastatin. A detailed overview of the steps is given in the Examples.

### EXAMPLES

### General Materials and Methods

Standard procedures were carried out as described elsewhere (Sambrook, J. et al. (1989), Molecular cloning: a laboratory manual, 2nd Ed., Cold Spring Harbor Laboratory Press, Cold Spring Harbor, N.Y.). DNA was amplified using the proofreading polymerases Turbo-Pfu-Polymerase or Herculase (Stratagene, The Netherlands), following the manufacturers protocol, while the verification of constructed strains and plasmids was achieved by using Taq polymerase Restriction enzymes were from Invitrogen or New England Biolabs. For routine cloning, Escherichia coli strains Top10 and DH10B (Invitrogen) were employed. Verification of the constructed plasmids was carried out by restriction analysis and subsequent sequencing (Seqlab GmbH, Goettingen, Germany). The filamentous fungus Aspergillus terreus strain ATCC20542 was used as a lovastatin producer in feeding experiments and served as a basis for genetic engineering trials (described below).

Extraction and HPLC analysis: freeze dried samples were extracted with 1 ml of methanol. To this end, 1 ml methanol was added to the freeze-dried material, followed by vortexing at maximal speed during at least 1 minute per sample. Care was taken that all material in the Eppendorf tube was brought into suspension, and that no clumps remained during extraction. The tubes were centrifuged at 13,000 rpm for 5 minutes, and the clear supernatant was transferred to HPLC sample tubes. HPLC analysis conditions were as follows:

| | |
|---|---|
| Eluens: | 60% acetonitril in milliQ water |
| Gradient: | No gradient |
| Column: | Waters XTerra RP18 |
| Column Temp: | room temperature |
| Flow: | 1 ml/min |
| Injection volume: | 10 μl |
| Tray Temp: | room temperature |
| Instrument: | Waters Alliance 2695 |
| Detector: | Waters 996 Photo Diode Array |
| Wavelength: | 238 nm |
| Retention times: | Lovastatin 3.8 min |
| | Simvastatin 4.7 min |

### Example 1

### Methylmalonate and Methylmalonyl-CoA are not Produced by Wild Type Aspergillus terreus

Conidiospores or Aspergillus terreus strain ATCC20542 (or strains derived thereof by mutation and selection for higher productivity, preferably in either of the recipes as stated below) are inoculated at 10E5-10E6 conidia/ml in a lovastatin production medium containing (g/l): dextrose, 40; CH₃COONH₄, 2.2; Na₂SO₄, 4; KH₂PO₄, 3.6; K₂HPO₄.3H₂O, 35.1; trace elements solution (citric acid.H₂O, 150; FeSO₄.7H₂O, 15; MgSO₄.7H₂O, 150; H₃BO₃, 0.0075; CuSO₄.5H₂O, 0.24; CoSO₄.7H₂O, 0.375; ZnSO₄.7H₂O, 1.5; MnSO₄.H₂O, 2.28; CaCl₂.2H₂O, 0.99), 10 (ml/l) (WO 98/037179). The culture is incubated at 28° C. in an orbital shaker at 220 rpm for 144-168 hours. At the end of the fermentation, the mycelium is removed by centrifugation or filtration and the mycelium is washed with physiological salt. Both the mycelium and the medium are assayed for malonate, malonate CoA, methyl butyrate, lovastatin, methylmalonate, methylmalonate-CoA, 2,2-dimethylbutyrate and simvastatin formed by LC-MS methods well known in the art. Only the first four compounds could be detected, confirming that A. terreus cannot synthesize methylmalonate de novo.

### Example 2

### Simvastatin Formation by Aspergillus terreus ATCC20542 fed with 2,2-dimethylbutyrate

Aspergillus terreus was cultivated as described in example 1. After a precultivation of 48-96 hours the cultures were diluted in fresh medium at a 1:10 ratio. Additionally, 0, 0.1 and 1.0 g/l of either methylmalonate or 2,2-dimethylbutyrate N-acetylcysteamine was added in the medium. The cultures were incubated in a horizontal shaker for 96-168 hours. The supernatant of the medium was subsequently separated from the cells and both the cells and medium were analyzed for lovastatin and simvastatin. Besides lovastatin, also simvastatin could be detected, but only in the cultivations were the 2,2-dimethylbutyrate precursors were added. In these simvastatin was present typically at 1/50^{th} of the lovastatin level.

### Example 3

### Metabolic Engineering Aspergillus terreus ATCC20542 with Malonate Synthase to Produce Methylmalonate-CoA

The malonyl-CoA synthetase gene from Rhizobium sp. (GenBank entry number H75771.1) was PCR amplified and cloned under control of the Aspergillus nidulans gpdA promoter and subsequently integrated into the genome of A. terreus. Standard fungal transformation technology was applied with amdS co-selection in order to screen for positive transformants (Ruiz-Diez, B., J. Appl. Microbiol. 2002, 92, pages 189-195). The transformants with the malonate synthase expression cassette integrated stably in the genome were selected with colony PCR. The transformants were cultivated as described in examples 1 and 2, and additionally, methylmalonate was added to the medium. After sample processing methylmalonyl-CoA could be detected.

### Example 4

### Metabolic Engineering Aspergillus terreus ATCC20542 with Propionyl-CoA Synthetase and Propionyl-CoA Carboxylase to Produce Methylmalonate-CoA

The propionyl-CoA synthetase (GenBank entry number R88078.1) and propionyl-CoA carboxylase (GenBank entry number AL939113) genes from Escherichia coli K12 and Streptomyces coelicolor, respectively, were PCR amplified and cloned under control of the Aspergillus nidulans gpdA promoter (GenBank entry number M19694) and subsequently integrated into the genome of A. terreus. Standard fungal transformation technology was applied with either amdS or hygromycin B as co-selection in order to screen for positive transformants. The transformants with the both expression cassettes integrated stably in the genome were selected with colony PCR. These were cultivated as described in examples 1 and 2, and additionally, propionate was added to the medium. After sample processing methylmalonate-CoA could be detected.

### Example 5

### In Vitro Conversion of Malonyl-CoA and Methylmalonyl-CoA into Lovastatin and Simvastatin

Aspergillus terreus was cultivated as described in example 1 for two days at 28° C. The cells were washed, freeze-dried and cell-free extracts were obtained. To assess the synthetic capacity for both statins the following assays were performed (summarized in the Table below):

After incubation for 1-24 hours the samples were analyzed. No products could be detected in reaction 1, the control. Lovastatin and simvastatin formation was readily visible in reactions 2 and 3, although the lovastatin formation rate was higher than the simvastatin formation rate. In samples 4 and 5 only traces could be detected due to the intracellular levels of monacolin J still present in the cells.

This result demonstrates that the enzyme set of Aspergillus terreus is capable of producing simvastatin when the right substrates are available.

| | Reaction nr. | | | | |
|---|---|---|---|---|---|
| | 1 | | | | |
| Ingredients | Control | 2 | 3 | 4 | 5 |
| 5 μl 500 mM Tris•HCl pH 7.5 | X | X | X | X | X |
| 5 μl monacolin J | | X | X | | |
| 5 μl 3 mM malonyl-CoA | | X | | X | |
| 5 μl 3 mM methylmalonyl-CoA | | | X | | X |
| 5 μl 3 mM acetyl-CoA | X | X | X | X | X |
| 5 μl 10 mM S-adenosylmethionine | X | X | X | X | X |
| 5 μl 1 mM NADPH | X | X | X | X | X |
| 5 μl 1 mM ATP | X | X | X | X | X |
| 10 μl CFE | X | X | X | X | X |
| H₂O (μl) | 15 | 5 | 5 | 10 | 10 |

### Example 6

### In Vitro Production of 2-methylbutyrate and 2,2-dimethylbutyrate Using LovF Polyketide Synthase

Construction of pSIMVA1 (pENTR/SD/D-Topo-LDKS) & pSIMVA2 (pET-DEST42-LDKS).

The lovF gene (GenBank number AAD34559.1) encoding the LDKS protein from Aspergillus terreus was PCR amplified by using oligo's directly surrounding the Open Reading Frame (ORF), using cDNA from Aspergillus terreus ATCC20542 grown under lovastatin producing conditions. The resulting 5 kb DNA fragment was purified from the agarose gel and subsequently used for cloning into pENTR/SD/D-Topo (Invitrogen Kit), following the manufacturers protocol, yielding pSIMVA1. The so obtained Gateway Entry vector (Gateway technology, Invitrogen, The Netherlands) was recombined with the Destination vector pET-DEST42 according to the manufacturers protocol, yielding the E. coli expression vector pET-DEST42-LDKS, or pSIMVA2. Sequences of both pSIMVA1 and pSIMVA2 were verified by DNA sequencing.

Recombinant Production of the Polyketide Synthase LovF in E. coli BL21 Star (Invitrogen, The Netherlands)

Plasmid pSIMVA2 and the pREP4-gsp plasmid (which encodes for a P-Pant-Transferase that modifies the ACP moieties, Mootz, H. D. et al. Proc. Natl. Acad. Sci. 2000, 97, pages 5848-5853) were transformed in E. coli BL21 Star cells. Both plasmids could be co-transformed because they harbor different resistance markers (for ampicillin and kanamycin, respectively). The resulting strain was used for recombinant production of the polyketide synthase LovF. Typically, 1 Liter 2YT (0.1 mg/mL Ampicillin) rich medium was inoculated with 10 mL of overnight culture BL21Star/pSIMVA2 grown in the same medium. Cultures were grown at 37° C. until an OD₆₀₀ of 0.5-0.7 was reached. Protein production was induced with 0.5 mM IPTG, and cells were grown for 4 hours at 30° C. The so obtained LovF protein was used for in vitro experiments. In most cases, the E. coli cells were lysed by sonicating cell suspensions in 50 mM phosphate buffer pH 6.8, 0.3 mM NaCl, 20% glycerol, 5 mM DTT, +/−1 mM ETDA, 1*Complete Protease inhibitor mix (Roche Diagnostics, Germany). After removing the cell debris by centrifugation, the obtained CFE was used for the experiments. Alternatively, we enriched the LovF protein by applying the CFE (lysed in buffer without EDTA) onto a Ni-NTA column. Due to the C-terminal HisTag present the enzyme binds at the matrix and can be eluted with 200 mM imidazole. Using ultra filtration, the enzyme can be concentrated to millimolar concentrations and subsequently used within enzyme assays.

In Vitro 2-methylbutyrate and 2,2-dimethylbutyrate-Formation Assay of the Polyketide Synthase LovF.

The activity of the LovF polyketide synthase was verified by screening for 2-methylbutyrate in an in vitro reaction. In a total volume of 1 mL of 50 mM phosphate buffer pH 6.8, 10-100 micromolar LovF (alternatively 100 microliter CFE containing LovF) was incubated with 1 mM Malonyl-CoA, 1 mM S-adenosylmethionine, 5 mM NAPDH, 5 mM DTT, 1 mM Acetyl-CoA. The reaction was carried out for 1 hour at 25 C. After completion, the polyketide was extracted with ethyl acetate (5% acetic acid). The organic phase was collected and the solvent was removed in vacuum (SpeedVac). Finally, the product was dissolved again in a small volume (1050 microliter methanol or acetonitrile). Analysis of the products 2-methylbutyrate and 2,2-dimethylbutyrate was performed with LC-MS/MS and NMR. 2,2-Dimethylbutyrate was typically present at 1% of the 2-methylbutyrate level.

### Example 7

### Improved In Vitro Production of 2,2-dimethylbutyrate Using an Engineered LovF Polyketide Synthase

### Construction of Plasmids

The fungal polyketide synthase LovF is composed of the following domains in the order:
KS-AT-DH-MT-KR-ER-ACP
   (KS=ketosynthase; AT=acyltransferase; DH=dehydratase; MT=methyltransferase; KR=ketoreductase; ER=enoylreductase; ACP=acyl carrier protein)

Based on this protein, we engineered two new hybrid proteins that both showed (increased) activity to form 2,2-dimethylbutyrate. The first hybrid PKS, referred to as Hybrid 1, was constructed by exchanging the acyltransferase domain (AT) from LovF with the acyltransferase (AT) domain of deoxyerythronolide B synthase module 6 from Saccharopolyspora erythreae (GenBank Entry number AAA26495.1). The second hybrid PKS, referred to as Hybrid 2, was constructed by exchanging the MT-fragment of LovF against the homologous fragment MT of the HMWP1 gene of Yersiniabactin Synthetase from Yersinia pestis (GenBank number AAC69588.1). The latter MT domain produces a geminal dimethyl-group and due to that gives rise to 2,2-dimethylbutyrate synthesis. Engineering of Hybrid 1. Plasmid pSIMVA1 carrying the lovF gene of Aspergillus terreus was used as a template for inserting restriction sites which flank the AT domain. AT Boundary definitions were chosen by DNA sequence alignment (BLAST search, NCBI). Forward and reverse DNA oligonucleotides that contain SpeI and PacI sites, respectively, were synthesized and the Quickchange Mutagenesis kit (Stratagene) was used to insert the restriction sites into the lovF gene. The experimental procedure was carried out according to the manufacturers protocol. Subsequently the constructs were completely sequenced to exclude oligo and polymerase errors. In parallel, the deoxyerythronolide B synthase AT6 domain gene from Saccharopolyspora erythreae was PCR amplified with oligo's carrying SpeI and PacI sites as well and cloned into pCR-Blunt vectors (Invitrogen, The Netherlands). Oligo's and restriction sites were created in a way to ensure in frame cloning of the EryAT6 domain into the lovF gene. After the DNA sequence of pSIMVA3 (pCR-Blunt-Ery AT6) was verified, pSIMVA4 (pENTR-SD-D-Topo-LovF (EryAT6) was constructed by first removing lovastatin AT using SpeI/PacI and subsequently ligating SpeI/PacI treated EryAT6 into the lovastatin construct. The expression plasmid pSIMVA5 (pET-DEST42-LovF (EryA6) was constructed using the Gateway reaction employing the manufacturers protocol. Engineering of Hybrid 2. The construction of Hybrid 2 was achieved similarly to the set up of Hybrid 1. SpeI/PacI flankings of the MT LovF fragment were inserted by oligo's carrying these restriction enzyme sites using the Quickchange mutagenesis kit. Also, the MT fragment from the Yersinlabactin Synthetase hmwp1 gene from Yersinia pestis was amplified with the same flanking restriction enzymes. Both fragments were exchanged, yielding the plasmid pSIMVA6 (pENTR-SD-D-Topo-LovF (MT HMWP1)). The expression plasmid pSIMVA7 (pET-DEST42-LovF (MT HMWP1) was constructed using the Gateway reaction employing the manufacturers protocol.

Recombinant Production of Hybrid 1 and Hybrid 2 in E. coli Bl21 Star and In Vitro Detection of 2,2-dimethylbutyrate

Plasmid pSIMVA5 or pSIMVA7, respectively, and the plasmid pREP4-gsp (which encodes for a P-Pant-Transferase that modifies the ACP moleties, Mootz, H. D. et al., Proc. Natl. Acad. Sci. 2000, 97, pages 5848-5853) was transformed in Escherichia coli BL21 Star cells. The resulting strain was used for recombinant production of the polyketide synthase LovF. Typically, 1 Liter 2YT (0.1 mg/mL Ampicillin) rich medium was Inoculated with 10 mL of overnight culture grown in the same medium. Cultures were grown at 37° C. until an OD₆₀₀ of 0.5-0.7 was reached. Protein production was induced with 0.5 mM IPTG, and cells were grown for 12-16 hours at 22° C. The so obtained LovF protein was used for in vitro experiments. In most cases, we lysed the E. coli cells by sonicating cell suspensions in 50 mM phosphate buffer pH 6.8, 0.3 mM NaCl, 20% glycerol, 5 mM DTT, +/−1 mM ETDA, 1*Complete Protease inhibitor mix (Roche Diagnostics, Germany). After removing the cell debris by centrifugation, the so obtained CFE was used for the experiments. Alternatively, we enriched the LovF protein by applying the CFE (lysed in buffer without EDTA) onto a Ni-NTA column. Due to the C-terminal HisTag present the enzyme binds at the matrix and can be eluted with 200 mM imidazole. In contrast to the wild type proteins, the hybrid enzymes should not be washed with imidazole concentrations higher than 5 mM. This is due to a lower affinity of the hybrid enzymes towards the Ni-NTA resins. Most likely, changes in the protein conformation result in a less accessible Hexa-Histidine affinity tag. Using ultra filtration, the enzyme can be concentrated to concentrations 0.1-0.5 mM and subsequently used within enzyme assays. The enzyme assays for the hybrid enzymes were carried out in analogy to the WT polyketide synthase LovF assays (see example 6). The product 2,2-dimethylbutyrate was analyzed using LC-MS/MS and NMR.

### Example 8

### In Vivo Production of Simvastatin Using Aspergillus terreus

### Construction of an Expression Cassette P_{gpdA}-LovF/Hybrid PKS-T_{penDE}

For the construction of a vector harboring the P_{gpdA}-LovF/Hybrid PKS-T_{penDE} cassette, the multisite Gateway technology (Invitrogen, The Netherlands) was employed. The methods used for the three PKS (LovF WT and the two Hybrid PKS) were equal. Therefore, the promoter of the gpdA gene of Aspergillus nidulans (GenBank Entry number M19694) was PCR-amplified using oligonucleotides with incorporated 5′-attB4 and 3′-attB1 sites. Recombination with the plasmid pDONRP4-P1R led to the ENTRY vector with flanking attL4 and attR1 sites. The LovF/Hybrid PKS genes were already cloned in ENTR vectors with flanking attL1 and attL2 sites and could be used directly for the multiple site gateway reactions. The terminator of the acyltransferase (penDE) gene from the penicillin biosynthetic pathway from Penicillium chrysogenum (GenBank Entry number 4379346) was PCR amplified with oligo nucleotides harboring 5′-attB2 and 3′-attB3 sites. Recombination with the vector pDONRP2R-P3 led to the ENTR vector with flanking attR2 and attR3 sites. These three ENTR vectors were then incubated with the destination vector pDEST-R4R3 in the LR-Clonase reaction, yielding the expression vectors pSIMVA8 (for LovF WT), pSIMVA9 (for Hybrid 1) and pSIMVA10 (for Hybrid2). The expression cassettes were finally sequenced to exclude sequence errors due to the PCR polymerase or the Gateway recombination reactions.

Random Integration of the Expression Cassettes of pSIMVA8-10 into as Aspergillus terreus Strain

Plasmids pSIMVA8-10 were transformed in Escherichia coli Top10 cells (Invitrogen, The Netherlands) and large-scale plasmid isolation from 100 mL overnight culture in rich media (2YT+100 μg/mL Ampicillin) was performed to yield 200 μg DNA from each plasmid. The P_{gpdA}-LovF/Hybrid PKS-T_{penDE} cassette was cut out of the plasmid backbones by using the proper restriction enzymes, such as XhoI/AscI or SpeI. The cassettes were gel-purified. Per transformation 5-10 μg DNA cassette was used, and as selection marker, the phleomycin resistance gene (Punt, P. J. et al. Methods of Enzymology 1992, 216, p. 447-457) under the control of the gpdA promoter was co-transformed into the lovF deficient Aspergillus terreus strain ATCC 20542 according to Ruiz-Diez, B., J. Appl. Microbiol. 2002, 92, p. 189-195. Alternatively, a lovF deficient Aspergillus terreus strain can be used, in which no competing pathway (e.g. lovastatin production) is present. This can be done separately, or either, in combination with the methylmalonyl-CoA synthesis pathways as described in Examples 3 and 4.

Cultivation of Transformed Aspergillus terreus Strains

Phleomycin resistant Aspergillus terreus colonies were chosen and further screened for the LovF or the hybrid PKS genes stably integrated in the genome by PCR and Southern blotting techniques. Positive candidates that harbored both the phleomycin resistance gene and the integrated PKS were then grown as described in example 1, with either 0.5 mM methylmalonate (in case of malonyl-CoA synthetase integration) or 1 mM propionate (in case of propionyl-CoA synthetase, carboxylase integration).

As a result, we identified:
Aspergillus terreus strains in which the integration of the WT lovF gene led to a full restorage of the Lovastatin production; and
both integrated hybrid PKS 1 and 2 led to the production of simvastatin.

Less lovastatin was found when the hybrid PKS genes were in a LovF deficient strain integrated. These results were verified and proven by a combination of LC-MS/MS and NMR techniques.

1. Method for the fermentative production of simvastatin using a microorganism harboring the Lov-biosynthetic gene cluster or a part thereof or genes homologous to said Lov-biosynthetic gene cluster.

2. Method according to claim 1 wherein the microorganism is either a prokaryote or a eukaryote.

3. Method according to claim 2 wherein the prokaryote is chosen from the group consisting of Bacillus amolyquefaciens, Bacillus subtilis and Escherichia coli.

4. Method according to claim 2 wherein the eukaryote is chosen from the group consisting of Aspergillus nidulans, Aspergillus terreus, Aspergillus niger, Penicillium citrinum, Penicillium brevicompactum, Penicillium chrysogenum, Monascus ruber, Monascus purpurea, Saccharomyces cerevisiae and Kluyveromyces lactis.

5. Method according to claim 1 anyone of the preceding claims wherein 2,2-dimethylbutyric acid or a salt or ester, thereof is added.

6. Method according to claim 1 wherein methylmalonic acid or a salt or ester thereof is added.

7. A microorganism harboring the Lov-biosynthetic gene cluster or a part thereof or genes homologous to said Lov-biosynthetic gene cluster equipped with a methylmalonyl-CoA biosynthesis pathway.

8. A DNA sequence with >30% identity to the lovastatin biosynthetic gene cluster that is being used in 2,2-dimethylbutyrate or simvastatin production.

9. A protein sequence with >30% identity to the lovastatin biosynthetic proteins that are being used in 2,2-dimethylbutyrate or simvastatin production.
